(19) United States
(12) Patent Application Publication
(10) Pub. No.: US 2011/0092456 A1
(43) Pub. Date: Apr. 21, 2011

(54) Methods of increasing cAMP levels and uses thereof

(76) Inventor: Yochai Birnbaum, Houston, TX (US)

(21) Appl. No.: 12/589,153

(22) Filed: Oct. 19, 2009

(51) Int. Cl. A61K 31/675; A61K 31/4985; A61K 31/40; A61K 31/403; A61K 31/4439; A61K 31/47; A61K 31/4402; A61K 31/4166; A61K 31/435; A61K 31/404; A61K 31/366; A61P 9/10

(52) U.S. Cl. 514/81; 514/249; 514/423; 514/412; 514/342; 514/312; 514/334; 514/392; 514/277; 514/419; 514/460

(57) ABSTRACT

The present invention shows that cilostazol, a phosphodiesterase 3 inhibitor has additional beneficial effects to atorvastatin on myocardial remodeling by inducing and preserving eNOS phosphorylation. The present invention demonstrates a cardioprotective effect of Cilostazol indicating the therapeutic potency of this drug. In addition, the present invention demonstrates that the additional effect of Cilostazol and atorvastatin therapy against ischemia injury is due to the augmentation of phosphatodylicositol 3-kinase/AKT (PI3-/AKT), PKA and p-eNOS signaling.

### CROSS-REFERENCE TO RELATED APPLICATION

This application claims benefit of priority under 35 U.S.C. §120 of international application PCT/US2008/005026, filed Apr. 18, 2008 which claims benefit of priority under 35 U.S.C. §119(e) of provisional U.S. Ser. No. 60/925,342, filed Apr. 19, 2007, now abandoned.

### BACKGROUND OF THE INVENTION

1. Field of the Invention

The present invention relates to the fields of cardiology, atherosclerosis and cell signaling. Specifically, the present invention describes that activation of protein kinase A by increasing intracellular levels of cAMP augments the pleiotropic effects of statins and thiazolidinediones, including protection against ischemia-reperfusion injury, anti-inflammatory and anti-atherosclerosis effects.

2. Description of the Related Art

Studies have shown that hydroxymethyl glutaryl coenzyme A reductase inhibitors (statins), administered either before myocardial ischemia or immediately after reperfusion reduce myocardial infarct size (infarct size). It has been suggested that the protective effect of statins is mediated by activation of phosphoinositol-3-kinase with subsequent activation of Akt, which activates endothelial nitric oxide synthase (eNOS) by phosphorylation at Ser-1177. Endothelial nitric oxide synthase activation is essential for this protective effect, as non-specific nitric oxide synthase (NOS) inhibitors blunt the infarct size-limiting effect of statins and statins do not reduce infarct size in eNOS^{-/-} mice. In addition, statins activate ecto-5-nucleotidase that generates adenosine. Adenosine has been shown to activate endothelial nitric oxide synthase. Inhibition of the adenosine receptors also have been shown to abrogate the infarct size-limiting effects of statins.

Endothelial dysfunction is recognized as an important process in the pathogenesis of atherosclerosis. Nitric oxide (NO) release by the endothelium regulates blood flow, inflammation and platelet aggregation, and consequently its disruption during endothelial dysfunction can decrease plaque stability and encourage the formation of atherosclerotic lesions and thrombi. Inhibitors of 3-hydroxy-3-methylglutaryl coenzyme A reductase (statins) are utilised in the prevention of coronary heart disease due to their efficacy at lowering lipid levels. However, statins may also prevent atherosclerotic disease by non-lipid or pleiotropic effects, for example, improving endothelial function by promoting the production of nitric oxide. There are various mechanisms whereby statins may alter nitric oxide release, such as inhibiting the production of mevalonate and isoprenoid intermediates, thereby preventing the isoprenylation of the small GTPase Rho, which negatively regulates the expression of endothelial nitric oxide synthase. Furthermore, statins may increase eNOS activity via post-translational at various sites by several kinases. The pathway most commonly cited is eNOS phosphorylation at Ser-1177 by the phosphatidylinositol 3-kinase/protein kinase Akt (PI3 K/Akt) pathway. However, this it has been reported that the activation of this pathway is short lived, as PTEN and SHIP-2 deactivate Akt. Protein kinase A (PKA) has been reported to activate eNOS by phosphorylation at both Ser-1177 and Ser-633. 8-Br-cAMP, an activator of PKA activates eNOS and improve endothelial function, similar to high dose atorvastatin. By increasing nitric oxide production, statins may interfere with atherosclerotic lesion development, stabilize plaque, inhibit platelet aggregation and improve blood flow and protect against ischemia. Therefore, the ability of statins to improve endothelial function through the release of nitric oxide may partially account for their beneficial effects at reducing the incidence of cardiovascular events. Moreover, eNOS activation by statins may contribute to vascular neogenesis and the creation of new blood vessels. Ability to augment eNOS phosphorylation by using combination of drugs may increase the anti-inflammatory and anti-atherosclerosis effects of statins.

In addition, PKA phosphorylates 5-lipoxygenase, preventing the enzyme from translocating into the peri-nuclear membrane and thus, inhibiting the production of leukotrienes, potent proinflammatory mediators, and increasing the production of 15-epi-lipoxins, potent anti-inflammatory mediators. Therefore, by activating PKA, the anti-inflammatory and anti-atherosclerosis effects of statins are potentiated at two sites: eNOS activation and the production of 15-epi-lipoxins. Moreover, prostacyclin induces vascular smooth muscle cell differentiation by activation by PKA. Atorvastatin increases tissue levels of prostacyclin, the combination of PKA activator with statins may augment this favorable effect. As PKA is activated by cAMP, increasing cAMP levels activates PKA and thus, augment the effects of statins.

However, when statins are administered orally, high doses are needed to achieve maximal cardioprotection. A 3-day pretreatment with oral atorvastatin (ATV) at 10 and 75 mg/kg/d reduces infarct size, whereas at 2 mg/kg/d atorvastatin alone does not affect infarct size. In another study, 3-day pretreatment with atorvastatin at 1 mg/kg/d did not affect myocardial levels of P-eNOS or calcium dependent NOS activity and did not limit infarct size. Thus, in order to achieve maximal protection, high-doses of statins are needed. Although blood levels of atorvastatin 16 hours after the third dose of atorvastatin (10 mg/kg/d) in the rat are comparable to those seen in humans treated with atorvastatin 80 mg/d, not all patients can tolerate maximal doses of statins. Moreover, the myocardial protective effect of statins may decay over time due to activation of phosphatase and tensin homolog deleted from chromosome 10 (PTEN), which inhibits the phosphorylation of Akt. Thus, it is warranted to explore means of enhancing the protective effect by use of drug combinations that may act on critical steps of the signaling pathway.

Further, clinicians and researcher have been frustrated, for a long time, because although the microvascular complications of diabetes mellitus can be altered by tight glycemic control, the macrovascular complications seem not to respond to such regimens. Although statins have been shown to reduce the risk of cardiovascular disease in diabetic patients, cardiovascular mortality and morbidity remain significantly higher in diabetic than in non-diabetic patients. Clinical studies have suggested that pioglitazone reduces cardiovascular complications in diabetic patients. Pioglitazone protects the heart against ischemia-reperfusion injury and reduces infarct size in the rat. The mechanism of protection involves upregulation of prostaglandin production through cytosolic phospholipase A₂ (cPLA₂) and cyclooxygenase-2 (COX2). Thiazolidinediones may activate endothelial nitric oxide synthase (eNOS) [67-71]. However, a 3-day pretreatment in the rat, pioglitazone (at 10 mg/kg/d) does not increase eNOS phosphorylation at Ser-1177 and does not augment calcium dependent NOS activity. Gonon et al reported that intraperitoneal rosiglitazone, administered 45 min before ischemia, attenuates myocardial stunning following ischemia-reperfusion in wild type, but not eNOS^{-/-} mice. COX2 inhibition completely blocks the infarct-size limiting effect of oral pioglitazone.

In addition, a three-day pretreatment with pioglitazone increases myocardial levels of 6-keto-PGF1α (the stable metabolite of prostacyclin), 15-epi-lipoxin A₄ (a potent anti-inflammatory mediator), and 15-deoxy-PGJ₂ (the natural ligand of PPAR-gamma. All three mediators have anti-inflammatory, anti-atherosclerotic and myocardial protective effects.

It is unclear whether the myocardial protective effect of thiazolidinediones is PPAR-γ dependent or independent. Zhao et al reported that the cerebral infarct size limiting effect of 5-day intracerebroventricular infusion of pioglitazone before middle cerebral artery occlusion was blocked by GW9662, a PPAR-γ antagonist. On the other hand, Brunmair et al suggested that the inhibitory effects of thiazolidinediones on skeletal muscle mitochondrial fuel oxidation is immediate and PPAR-γ independent. Moreover, 15dPGJ₂ and non-thiazolidinedione PPAR-γ agonists do not have the same effect. Low-dose troglitazone, but not pioglitazone, activates ERK phosphorylation in renal tubule-derived cell lines; whereas pioglitazone, but not troglitazone, activates AMP-activated protein kinase. These effects are PPAR-γ independent. Whereas the activation of eNOS by rosiglitazone and ciglitazone in human umbilical vein endothelial cells (HUVEC) is PPAR-γ dependent, it has been shown that the increased production of 15dPGJ₂ by pioglitazone in HUVEC cells was not inhibited by GW9662, a PPAR-γ antagonist, suggesting that pioglitazone has a unique property to augment the production of prostaglandins by a PPAR-γ independent mechanism. The mechanisms of conferring neuroprotection by rosiglitazone and 15dPGJ₂ differ. As pioglitazone increases the production of 15dPGJ₂, both mechanisms are expected to occur after pioglitazone pretreatment.

The protective effect of pioglitazone occurs rapidly, as pioglitazone reduced infarct size, when administered just prior to coronary artery occlusion in an isolated heart model. Intravenous troglitazone, injected 15 min before coronary artery occlusion, limits infarct size in the dog. Neuroprotection was seen also when rosiglitazone and 15dPGJ₂ were given after permanent middle cerebral artery occlusion. Previously, 3-day pretreatment with pioglitazone limits infarct size in the rat. Three-day pretreatment with pioglitazone also reduces cerebral infarct size in the rat. Ito et al showed that 7-day pretreatment with pioglitazone at limits infarct size. On the other hand, 8 weeks of treatment with troglitazone failed to limit infarct size in the pig. Thus, it might be that the myocardial protective effect of thiazolidinediones in general, and/or pioglitazone, may decay over time, as has been reported that the myocardial protective effect of atorvastatin is not seen after 7 days of pretreatment. Moreover, it might be that the mechanisms of protection may change with the duration of therapy, as has been shown for statins. For example, in isolated rat heart ERK inhibition blocks the protective effect of pioglitazone only when administered at reperfusion, but not before ischemia. On the other hand, rosiglitazone reduces infarct size and suppresses ERK phosphorylation, without altering Akt phosphorylation.

Protein kinase A (PKA) is also involved in myocardial protection by ischemic preconditioning. Previously, pioglitazone has been demonstrated to increases PKA. PKA phosphorylates 5-lipoxygenase, leading to the production of 15-epi-lipoxin A₄, a strong anti-inflammatory mediator. In addition, PKA activates eNOS by phosphorylation at Ser-1177 and Ser-633 in an Akt-independent way. GLP-1 activates adenylyl cyclase, and thus, increases tissue levels of camp. cAMP activates several enzymes, including PKA. GLP-1 limits myocardial infarct size. JANUVIA (sitagliptin phosphate), a selective dipeptidyl peptidase IV inhibitor, increases GLP-1 levels, thus should lead to an increase in cAMP levels and PKA activation.

Cilostazol (CIL) is a phosphodiesterase 3 inhibitor, increasing cellular levels of cyclic AMP (cAMP), with antiplatelet and vasodilatatory properties and is approved in the U.S. for treatment of patients with intermittent claudication symptoms related to peripheral arterial disease.

The prior art is deficient in means of enhancing cardioprotection against ischemic injury by use of drug combinations that act on critical signaling pathway steps. Specifically, the prior art is deficient in the knowledge of whether combining statins with cilostazol or dipeptidyl peptidase-4 inhibitors with PPAR-gamma ligands would mediate cardioprotection against ischemic injury. The prior art also lacks the understanding whether combining statins with dipeptidyl peptidase-4 inhibitors or PPAR-gamma ligands would augment and sustain the cardioprotective effects of stains thereby allowing for the use of lower doses of statins. The present invention full fills this long lasting need in the art.

### SUMMARY OF THE INVENTION

The present invention is directed to a method of reducing ischemia-reperfusion injury in an individual in need of such treatment consisting of administering a compound or a combination of compounds, in an amount effective in increasing intracellular levels of cyclic adenosine monoposphate, thereby reducing ischemia-reperfusion injury in the individual.

The present invention describes a method of protection against ischemia-reperfusion injury in an individual in need of such treatment consisting of administration of a phosphodiesterase type three inhibitor, in an amount, effective to activate endothelial nitric oxide synthase, thereby inducing myocardial protection in the individual.

The present invention also describes a method of augmenting the cardioprotective effects of a HMG-CoA reductase inhibitor in an individual consisting of co-administration of pharmacologically effective amounts of HMG CoA reductase inhibitor with a phosphodiesterase 3 inhibitor, where the co-administration augments the cardioprotection in the individual.

The present invention further describes a method of preventing attenuation of the myocardiac protective effects of HMG-CoA reductase inhibitor, in an individual consisting of co-administration of pharmacologically effective amounts of HMG CoA reductase inhibitor with a phosphodiesterase 3 inhibitor, wherein the co-administration prevents the attenuation of the myocardiac protective effects of HMG-CoA reductase inhibitor in said individual.

The present invention is also directed to a method of augmenting the cardioprotective effects of a HMG-CoA reductase inhibitor in an individual consisting of co-administering pharmacologically effective amounts of a HMG CoA reductase inhibitor with a compound effective in increasing intracellular levels of cyclic adenosine monoposphate, wherein said co-administration augments the cardioprotection in the individual.

The present invention is further directed to a method of attenuating inflammation in an individual in need of such treatment consisting of administering a compound or a combination of compounds, in an amount effective in increasing intracellular levels of cyclic adenosine monoposphate, thereby attenuating inflammation in said individual.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows infarct size (as a percentage of the AR) in the rats. Overall there were significant differences among groups (p<0.001). atorvastatin alone did not affect infarct size. In contrast, infarct size in the cilostazol alone and atorvastatin+cilostazol groups was significantly smaller than in the control group. infarct size in the atorvastatin+cilostazol group was significantly smaller (p≦0.002) from the other three groups). (*−p<0.001 versus the control group).

FIG. 2 shows mean heart rate at baseline, before coronary artery occlusion, 25 min of ischemia and 20 min of reperfusion. Overall there was a significant group (p<0.001), as well as time (p<0.001) effect; However, the absolute differences among groups were small.

FIG. 3 shows mean blood pressure at baseline, before coronary artery occlusion, 25 min of ischemia and 20 min of reperfusion. Overall there was a significant group (p=0.011), as well as time (p<0.001) effect. However, the absolute differences among groups were small.

FIG. 4 shows PKA activity. Overall, there was a significant difference among groups (p<0.001). *−p<0.05 versus control; #−p<0.05 versus atorvastatin+cilostazol.

FIGS. 5A-5D show representative immunoblots and densitometric analyses of myocardial levels of total Akt (FIG. 5A and FIG. 5B) and total eNOS (FIG. 5C and FIG. 5D).

FIGS. 6A-6B show representative immunoblots (FIG. 6A) and densitometric analyses (FIG. 6B) of myocardial levels of Ser-473 P-Akt. * p<0.05 versus cont; † p<0.05 versus atorvastatin; ‡ p<0.05 versus cilostazol.

FIGS. 7A-7D show representative immunoblots and densitometric analyses of myocardial levels of Ser-1177 P-eNOS (FIG. 7A and FIG. 7B) and Ser-633 P-eNOS (FIG. 7C and FIG. 7D). * p<0.05 versus cont; † p<0.05 versus atorvastatin; ‡ p<0.05 versus cilostazol.

FIGS. 8A-8B show representative immunoblots (FIG. 8A) and densitometric analyses (FIG. 8B) of myocardial levels of PTEN. * p<0.05 versus cont; † p<0.05 versus atorvastatin.

FIG. 9 shows myocardial adenosine levels. Overall, there were significant differences among groups (p=0.002). atorvastatin alone (p=0.028) and cilostazol alone (p=0.025) caused a small increase in myocardial adenosine levels compared to the control group. Myocardial adenosine levels were significantly higher in the atorvastatin+cilostazol group than in the control group (p=0.001).

FIG. 10: Myocardial infarct 600 size (IS, % of the ischemic area at risk) in the different groups (protocol 1-3-day pretreatment). * p<0.05 vs. control; # p<0.05 vs. SIT+PIO; $ P<0.05 vs. SIT).

FIG. 11: Myocardial infarct size (IS, % of the ischemic area at risk) in the different groups (protocol 2-14-day pretreatment). * p<0.001 vs. control; # p<0.001 vs. SIT+PIO; $ P=0.014 vs. SIT+PIO).

FIG. 12A-12D show an aspect of the relationship between cAMP levels and PKA activity. FIG. 12A: Myocardial cAMP levels. * p<0.05 vs. control. FIG. 12B: Myocardial PKA activity. FIG. 12C: Myocardial cPLA₂ activity. FIG. 12D: Myocardial COX2 activity. * p<0.001 vs. control; # p<0.005 vs. ST+PIO.

FIG. 13A-13C shows the effect of various agents on eicosanoid levels. FIG. 13A: Myocardial 6-keto-PGF_{1α} levels. FIG. 13B: myocardial 15dPGJ₂ levels. FIG. 13C: Myocardial 15-epi-lipoxin A₄ levels. * p<0.001 vs. control.

FIG. 14A-14D shows the effects of ischemia-reperfusion on total eNOS levels. Samples of immunoblots (FIG. 14A) and densitometric analysis of total eNOS (FIG. 14B), Ser-1177 PeNOS (FIG. 14C), and Ser-633 PeNOS (FIG. 14D). * p<0.001 vs. control no IR. # p<0.001 vs. control IR; $ p<0.001 H-89 vs. no H-89.

FIG. 15A-15C show the effects of ischemia-reperfusion without or with PIO or SIT on total CREB levels. Samples of immunoblots (FIG. 15A) and densitometric analysis of total CREB (FIG. 15B) and P-CREB (FIG. 15C). * p<0.001 vs. control no IR. # p<0.001 vs. control IR; $ p<0.001 H-89 vs. no H-89.

### DETAILED DESCRIPTION OF THE INVENTION

The present invention shows that cilostazol at 20 mg/kg/d reduced infarct size and augmented myocardial levels of Ser-473 P-Akt and Ser-633 P-eNOS and Ser-1177 P-eNOS. Atorvastatin at 2 mg/kg/d had a small effect on Ser-473 P-Akt, but did not increase myocardial Ser-1177 P-eNOS levels and did not affect infarct size. The combination of atorvastatin and cilostazol had a synergistic effect on infarct size-limitation and on myocardial levels of Ser-473 P-Ak, Ser-1177 P-eNOS and Ser-633 P-eNOS.

Activation of Akt and endothelial nitric oxide synthase by phosphorylation at Ser-473 and Ser-1177, respectively, are essential steps for mediating the myocardial protective effects of statins. Although most studies have suggested that P-Akt directly phosphorylates endothelial nitric oxide synthase at Ser-1177, other enzymes can also phosphorylate endothelial nitric oxide synthase at Ser-1177 (PKA, protein kinase G and AMP-activated protein kinase). By inhibiting phosphodiesterase 3, cilostazol would increase myocardial levels of cAMP and activates PKA, thus augmenting endothelial nitric oxide synthase activation by phosphorylation at both Ser-1177 and Ser-633. Thus, synergism between atorvastatin and cilostazol in endothelial nitric oxide synthase activation and myocardial protection could be expected. Indeed, cilostazol augmented endothelial nitric oxide synthase phosphorylation at both Ser-1177 and Ser-633 and this effect was augmented by co-administration of atorvastatin.

Hashimoto et al showed that cilostazol increased nitric oxide production in human aortic endothelial cells by upregulating endothelial nitric oxide synthase phosphorylation at Ser-1177 and dephosphorylation at Thr-495. They reported that cilostazol increases Akt phosphorylation at Ser-473, supporting findings that cilostazol activates Akt. However, they reported that endothelial nitric oxide synthase phosphorylation is blocked by both a PKA inhibitor and a phosphatidylinositol 3-kinase (PI3K) inhibitor, suggesting that both enzymes are mediating the activation of endothelial nitric oxide synthase by cilostazol. As endothelial nitric oxide synthase phosphorylation at Ser-633 is mediated by PKA and not by Akt, these results support that endothelial nitric oxide synthase activation by atorvastatin is mediated by both Akt and PKA.

The exact mechanisms of enhanced Akt phosphorylation by cilostazol are unknown; however, cilostazol may inhibit phosphatase and tensin homolog deleted from chromosome 10 (PTEN) activation by tumor necrosis factor-α. PTEN hydrolyzes phosphatidylinositol-3,4,5-trisphosphate to phosphatidylinositol-4,5-bisphosphate. As Akt phosphorylation is controlled by phosphatidylinositol-3,4,5-trisphosphate levels, PTEN affects the phosphorylation of Akt. The results of the present invention demonstrate that cilostazol alone or in combination with atorvastatin significantly reduce PTEN levels. Others reported that cilostazol inhibits the reuptake of adenosine similarly to dipyridamole and thus increasing interstitial adenosine levels. Adenosine has been shown to activate Akt and endothelial nitric oxide synthase. Statins activate ecto-5-nucleotidase and increase myocardial levels of adenosine. The atorvastatin-induced upregulation of endothelial nitric oxide synthase phosphorylation at Ser-1177 by Akt is completely blocked by theophylline, a non-selective adenosine receptor antagonist. Thus, a possible synergistic effect could be through augmentation of interstitial adenosine levels.

Cilostazol may protect against ischemia reperfusion injury. Liu et al showed that although intravenous cilostazol (1 mg/kg/min for 5 min before myocardial ischemia) does not affect myocardial infarct size in the rabbit, it potentiates the effect of preconditioning by 2 min of ischemia. SPT, an adenosine receptor blocker, abrogates the infarct size-limiting effect of cilostazol+preconditioning, suggesting that the effect is mediated by adenosine. However, cilostazol levels with their protocols were probably much higher than in the present invention. Lee et al showed that oral cilostazol (30 mg/kg) administered at 5 min and 4 h after completion of 2 hours of middle cerebral artery occlusion reduces brain infarct size in the rat. They also found that cilostazol augmented Akt phosphorylation. Oral cilostazol (30 mg/kg), but not aspirin (300 mg/kg) or clopidogrel (30 mg/kg) reduced brain infarct size in a rat model of 2 h of middle cerebral artery occlusion and 24 h of reperfusion. Wakida et al reported that cilostazol (30 mg/kg), administered intraperitoneally three times (at 12 h before, 1 h before and just after the induction of cerebral ischemia) reduced brain infarct size measured at 24 h in a mouse model of permanent middle cerebral artery occlusion. In contrast to the previous studies, the instant invention used a smaller dose of cilostazol administered once daily by oral gavage for 3 days before the induction of ischemia and found a significant protective effect, which could be further augmented by combining cilostazol with low dose atorvastatin.

Most patients with established or at high risk for cardiovascular disease are receiving multiple drugs including statins and antiplatelet agents, mainly aspirin. An adverse interaction between atorvastatin and aspirin was recently shown. When the cyclooxygenase is both acetylated by aspirin and S-nitrosylated by atorvastatin-induced inducible nitric oxide synthase (iNOS), the enzyme is inactivated, and the production of prostacyclin and 15-epi-lipoxin is decreased. By acetylation of cyclooxygenase-2 aspirin blunts the infarct size-limiting effects of atorvastatin, as do selective cyclooxygenase-2 inhibitors.

Cilostazol may be an effective substitute for aspirin in patients receiving statins since it has antiplatelet and vasodilatatory properties and is approved in the US for treatment of patients with intermittent claudication symptoms related to peripheral arterial disease. Studies have shown that cilostazol may be efficacious also in coronary artery disease.

In a study of 41 patients undergoing directional coronary atherectomy, cilostazol (200 mg/d) resulted in better 6-month angiographic outcomes than aspirin (250 mg/d). The minimal lumen diameter at follow-up was larger and the percent diameter stenosis was smaller in the cilostazol group. Intravascular ultrasound showed that the plaque area was smaller in the cilostazol group. In another small study (70 patients), cilostazol (200 mg/d) resulted in less in-stent stenosis than aspirin (81 mg/d) after successful coronary Palmaz-Schatz stenting (8.6% versus 26.8%). Similar findings were reported by Sekiya et al in 126 patients randomized to cilostazol, probucol, their combination or control after elective coronary stenting. Cilostazol (100 mg BID) reduced the incidence of 6-month restenosis rate (0% versus 20%; p<0.05) and target lesion revascularization (o % versus 16%; p=0.10), as compared to aspirin alone in 50 patients undergoing primary coronary stenting for ST elevation acute myocardial infarction. In this trial all patients received aspirin 81 mg tid for 6 months and the control group received ticlopidine for 1 month. Park et al reported that the 1-month clinical outcomes after coronary stenting were comparable between patients randomized to aspirin+ticlopidine versus aspirin+cilostazol. The Randomized Prospective Antiplatelet Trial of Cilostazol Versus Ticlopidine in Patients Undergoing Coronary Stenting (RACTS) trial showed that 9-month target lesion revascularization rate per patient was significantly lower in the cilostazol group (100 mg bid for 6 months, n=201) than in the ticlopidine group (250 mg bid for 1 month, n=196) (22.9% versus 32.7%, P=0.030) post-coronary stenting, although there was no significant difference in the composite incidence of death, myocardial infarction, stroke, and stent thrombosis between the 2 groups.

The Cilostazol for Restenosis Trial (CREST) reported that 6-month restenosis after successful coronary stenting was 22.0% in cilostazol (100 mg bid) treated patients (n=354) versus 34.5% in the control group (n=351) (P=0.002). In this trial all patients received clopidogrel for 30 days and aspirin for 6 months. Inoue et al reported that cilostazol, as compared to ticlopidine, suppressed platelet P-selectin (CD62P) and neutrophil Mac-1 (CD11b) induction and reduced restenosis following coronary stenting. Studies in animals showed that cilostazol suppresses atherosclerosis formation in low-density lipoprotein receptor (Ldlr)-null mice by suppressing superoxide and TNF-α formation, and thereby reducing NF-κB activation/transcription, VCAM-1/MCP-1 expressions, and monocyte recruitments. In the dog, cilostazol prevented coronary re-thrombosis following thrombolysis in a model of coronary artery thrombosis superimposed on high-grade stenosis.

The inhibitory effect of cilostazol on phosphatase and tensin homolog deleted from chromosome 10 (PTEN) activation may have an important effect when combined with statin therapy. Atorvastatin treatment increases myocardial PTEN levels. It has been suggested that PTEN upregulation after 7 days of oral atorvastatin treatment prevents Akt phosphorylation and blocked the infarct size-limiting effect of atorvastatin when given for 7 or 14 days. Thus, cilostazol may prevent this rapid decay in the protective effect.

Thus, cilostazol has a direct myocardial protective effect against ischemia-reperfusion injury, mediated by upregulation of P-Akt and P-eNOS. Atorvastatin and cilostazol have synergistic effect on Akt and endothelial nitric oxide synthase phosphorylation and infarct size limitation. The potential effect of cilostazol in preventing the decay in the protective effect of statins may be of value, especially with long-term treatment, as usually seen in the clinical setting.

In one embodiment of the present invention there is provided a method of reducing ischemia-reperfusion injury in an individual in need of such treatment consisting of administering a compound or a combination of compounds, in an amount effective in increasing intracellular levels of cyclic adenosine monoposphate, thereby reducing ischemia-reperfusion injury in said individual. Specifically, administration of the compounds or a combination of compounds activates protein kinase A. The activation of protein kinase A induces activation of eNOS via phosphorylation of Ser-633 and Ser-1177 residues of the endothelial nitric oxide synthase polypeptide. In general, the individual is at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy. Specifically, the treatment results in reduction of infarct size in said individual. In general, the compound is a dipeptidyl peptidase-4 inhibitor. Representative dipeptidyl peptidase-4 inhibitors include but are not limited to vildagliptin, sitagliptin or saxagliptin. Moreover, the compound may be a ligand for the peroxisome proliferator-activated receptor gamma. Particularly, the peroxisome proliferator-activated receptor gamma is a thiazolidinediones. Specifically, representative compounds include pioglitazone or rosiglitazone. Further, the combination of drugs administered is a combination of a dipeptidyl peptidase-4 inhibitor and a thiazolidinediones. A person having ordinary skill in this art could readily determine the optimal doses and routes of administration of the dipeptidyl peptidase-4 inhibitor and the thiazolidinedione.

In another embodiment of the present invention, there is provided a method of protection against ischemia-reperfusion injury in an individual in need of such treatment comprising administration of a phosphodiesterase type three inhibitor, in an amount effective to activate endothelial nitric oxide synthase, thereby inducing myocardial protection in the individual. In general, the phosphodiesterase type 3 inhibitor activates endothelial nitric oxide synthase activation via phosphorylation of Ser-633 and Ser-1177 residues of the endothelial nitric oxide synthase polypeptide. Moreover, the phosphorylation of Ser-633 and Ser-1177 residues of endothelial nitric oxide synthase involves activation of AKT and protein kinase A and suppression of PTEN activity. Additionally, the endothelial nitric oxide synthase activation may be due to suppression of adenosine reuptake into the cell. Specifically, the treatment may result in reduction of infarct size in the individual. In general, the individual is at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy. Specifically, the phosphodiesterase 3 inhibitor is cilostazol, amrinone, bucladesine, enoximone or milrinone.

In yet another embodiment of the present invention there is provided a method of augmenting the cardioprotective effects of a HMG-CoA reductase inhibitor in an individual comprising co-administration of pharmacologically effective amounts of HMG CoA reductase inhibitor with a phosphodiesterase 3 inhibitor, where the co-administration augments the cardioprotection in the individual. Specifically, the co-administration of pharmacologically effective amounts of HMG CoA reductase inhibitor and the phosphodiesterase 3 inhibitor lead to synergistic activation of endothelial nitric oxide synthase. The activation of endothelial nitric oxide synthase involves phosphorylation of Ser-633 and Ser-1177 residues of the endothelial nitric oxide synthase polypeptide. Also, the phosphorylation of Ser-633 and Ser-1177 residues of the endothelial nitric oxide synthase polypeptide involves activation of AKT and protein kinase A and a suppression of PTEN activity. Moreover, the activation of endothelial nitric oxide synthase may be due to suppression of adenosine reuptake. Specifically, the augmentation of myocardial protection is against ischemia-reperfusion injury of the coronary muscle. In general, the treatment results in reduction of infarct size in said individual. Moreover, the individual is at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy. A person having ordinary skill in this art could readily determine the optimal doses and routes of administration of the HMG-CoA reductase inhibitors. Particularly, representative HMG-CoA reductase inhibitors include but are not limited to atorvastatin, cerivastatin, fluvastatin, lovastatin, mevastatin or pitastatin. Specifically, representative phosphodiesterase 3 inhibitors include cilostazol, amrinone, bucladesine, enoximone or milrinone. A person having ordinary skill in this art could readily determine the optimal doses and routes of administration of the phosphodiesterase 3 inhibitors.

In yet still another embodiment of the present invention there is provided a method of preventing attenuation of the myocardiac protective effects of HMG-CoA reductase inhibitor, in an individual comprising co-administration of pharmacologically effective amounts of HMG CoA reductase inhibitor with a phosphodiesterase 3 inhibitor, where the co-administration prevents the attenuation of the myocardiac protective effects of HMG-CoA reductase inhibitor in said individual. Specifically, the protective effects of the HMG-CoA reductase inhibitor are due to the activation of the endothelial nitric oxide synthase. Moreover, the prevention of attenuation of the protective effects is due to phosphodiesterase 3 inhibitor preserving the activation of endothelial nitric oxide synthase. Specifically, the phosphodiesterase 3 inhibitor preserves activation of endothelial nitric oxide synthase via activating AKT and protein kinase A. Additionally, the phosphodiesterase 3 inhibitor preserves activation of endothelial nitric oxide synthase via suppression of PTEN activation. Also, the phosphodiesterase 3 inhibitor preserves activation of endothelial nitric oxide synthase via inhibition of adenosine reuptake. Specifically, the treatment results in reduction of infarct size in said individual. The HMG-CoA reductase inhibitors are atorvastatin, cerivastatin, fluvastatin, lovastatin, mevastatin or pitastatin. Moreover, the phosphodiesterase 3 inhibitor is cilostazol, amrinone, bucladesine, enoximone or milrinone.

In still yet another embodiment of the present invention there is provided a method of augmenting the cardioprotective effects of a HMG-CoA reductase inhibitor in an individual comprising of co-administering pharmacologically effective amounts of a HMG CoA reductase inhibitor with a compound effective in increasing intracellular levels of cyclic adenosine monoposphate, where the co-administration augments the cardioprotection in the individual. Specifically, the compound is an activator of protein kinase A. The co-administration of pharmacologically effective amounts of HMG CoA reductase inhibitor and the compound leads to a synergistic activation of endothelial nitric oxide synthase. Specifically, the activation of eNOS involves phosphorylation of Ser-633 and Ser-1177 residues of the endothelial nitric oxide synthase polypeptide. In general, the individual at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy. Moreover, the treatment results in reduction of infarct size in said individual. In general, the compound is a dipeptidyl peptidase-4 inhibitor. Specifically, representative dipeptidyl peptidase-4 inhibitors include vildagliptin, sitagliptin or saxagliptin. Furthermore, the compound is a ligand for the peroxisome proliferator-activated receptor gamma. Specifically, the peroxisome proliferator-activated receptor gamma is a thiazolidinediones. The thiazolidinedione may be, for example, pioglitazone or rosiglitazone. Moreover, the HMG-CoA reductase inhibitors are selected from the group consisting of atorvastatin, cerivastatin, fluvastatin, lovastatin, mevastatin and pitastatin.

In a related embodiment of the present invention there is a method of attenuating inflammation in an individual in need of such treatment consisting of administering a compound or a combination of compounds, in an amount effective in increasing intracellular levels of cyclic adenosine monoposphate, thereby attenuating inflammation in said individual. The inflammatory response is due to atherosclerosis or ischemia-reperfusion injury. Specifically, the administration activates protein kinase A. Moreover, the activation of protein kinase A induces activation of endothelial nitric oxide synthase via phosphorylation of Ser-633 and Ser-1177 residues of the endothelial nitric oxide synthase polypeptide. In general, the individual is at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy. Specifically, the treatment results in improved blood flow, reduction of infarct size in said individual and protection against ischemia. Additionally, the treatment results in prevention of plaque formation, attenuation of plaque inflammation and an increased plaque stability in said individual. Specifically, the compound is a dipeptidyl peptidase-4 inhibitor. Moreover, the dipeptidyl peptidase-4 inhibitor is vildagliptin, sitagliptin or saxagliptin. Additionally, the compound is a ligand for the peroxisome proliferator-activated receptor gamma. In general, the peroxisome proliferator-activated receptor gamma is a thiazolidinediones. Representative thiazolidinediones include pioglitazone or rosiglitazone.

In another related embodiment of the present invention the combination of compounds administered is a combination of a dipeptidyl peptidase-4 inhibitor and a thiazolidinedione. Moreover, the combination of compounds administered is a combination of a HMG-CoA reductase inhibitor and a thiazolidinedione.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Animal Care

The experimental designs and care of animals were conducted in accordance with ‘The Guide for the Care and Use of Laboratory Animals’ published by the US National Institutes of Health (NIH Publication No. 85-23, revised 1996). The male Sprague-Dawley rats were housed at a controlled temperature (24.5-25.0° C.).

### Example 2

### Drugs and Pretreatment

Crushed tablets of cilostazol (Pletal, Otsuka America Pharmaceuticals, Inc.) and atorvastatin (ATV) (Pfizer, US Pharmaceuticals) were used. cAMP-Dependent Protein Kinase A (PKA) Assay kit was purchased from Promega (Madison, Wis.). Adenosine 5′ triphosphate [γ32P] ATP 3000 Ci/mmol was purchased from Perkin Elmer (Waltham, Mass.). Monoclonal anti-eNOS antibodies were purchased from BD Bioscience (San Jose, Calif.) and monoclonal anti-β-Actin antibody from Sigma (St. Louis, Mo.). Anti-Akt antibodies, anti-Ser⁴⁷³ phosphorylated-Akt antibodies, and anti-Ser¹¹⁷⁷ phosphorylated-eNOS antibodies were purchased from Cell Signaling (Beverly, Mass.). Rats received 3-day pretreatment with: 1) water alone (sham); 2) atorvastatin (2 mg/kg/d); 3) cilostazol (20 mg/kg/d); or 4) atorvastatin (2 mg/kg/d) and cilostazol (20 mg/kg/d). Atorvastatin and cilostazol were administered by gastric gavage once daily.

### Example 3

### Infarct Size (Infarct Size) Surgical Protocol

The rat model of myocardial ischemia-reperfusion injury has been described in detail (Birnbaum, 2005 #215; Birnbaum, 2003 #198; Tavackoli, 2004 #214); On the fourth day, rats were anesthetized with intraperitoneal injection of ketamine (60 mg/kg) and xylazine (6 mg/kg). The animals were intubated and connected to an animal ventilator (Harvard Apparatus, Model 683, South Natick, Mass.) and ventilated using FIO₂ of 30%. The rectal temperature was monitored and body temperature was maintained between 36.7 and 37.3° C. with the aid of a heating lamp and heating pad. The left carotid artery was cannulated for monitoring heart rate and blood pressure, the chest was opened and a snare was placed around the left coronary artery to produce regional ischemia. Isofluorane (1-2.5% titrated to effect) was added after the beginning of ischemia to maintain anesthesia. The snare was released after 30 min ischemia and myocardial reperfusion was verified by change in the color of the myocardium. Subcutaneous 0.1 mg/kg buprenorphine was administered, the chest was closed and the rats were recovered from anesthesia. Four hours after reperfusion the rats were re-anesthetized, the coronary artery was reoccluded, 1.5 ml of Evan's blue dye 3% was injected into the right ventricle and the rats euthanized under deep anesthesia. Heart rate and mean blood pressure were noted at baseline (10 minutes after completion of surgery), just before coronary artery occlusion); at 25 minutes of ischemia; and at 20 minutes of reperfusion. The pre-specified exclusion criteria were lack of signs of ischemia during coronary artery ligation, lack of signs of reperfusion after release of the snare, prolonged ventricular arrhythmia with hypotension, and area at risk ≦10% of the LV weight.

### Example 4

### Determination of Area at Risk (AR) and Infarct Size

Hearts were excised and the left ventricle was sliced transversely into 6 sections. Slices were weighed and incubated for 15 minutes at 37° C. in 1% buffered (pH=7.4) 2,3,5-triphenyl-tetrazolium-chloride (TTC), fixed in a 10% formaldehyde and photographed in order to identify the AR (uncolored by the blue dye), the infarct size (unstained by TTC), and the non-ischemic zones (colored by blue dye). The area of AR and infarct size in each slice were determined by planimetry, converted into percentages of the whole for each slice, and multiplied by the weight of the slice and the results summed to obtain the weight of the myocardial AR and infarct size.

### Example 5

### Western Blot Analysis

Rats were treated as above, anesthetized and the hearts were removed and rinsed with cold PBS (pH 7.4), containing 0.16 mg/ml heparin to remove red blood cells and clots, Myocardial samples from the anterior left ventricular wall were frozen rapidly in liquid nitrogen and homogenized in RIPA lysis Buffer (Santa Cruz Biotechnology), centrifuge at 14,000 rpm for 10 min at 4° C. The supernatant was collected and the total protein concentration was determined using Lowry Protein Assay. The protein samples were subjected to SDS-PAGE with 4-20% gradient polyacrylamide gel and transferred to pure nitrocellulose membrane (0.45 μm) (Bio-Rad). After blocking with 5% skim milk in Tris-buffered saline, the membrane was incubated overnight at 4° C. with primary antibodies against eNOS, Akt, Ser¹¹⁷⁷ P-eNOS, Ser⁶³³ P-eNOS, Ser⁴⁷³ P-Akt, and PTEN, and secondarily with HRP-conjugated anti-mouse or anti-rabbit antibodies. The immunoblots were developed using ECL western Blotting Detection Reagent (Amersham). The protein signals were pictured by an image-scanner and analyzed using Image J software (National Institutes of health, USA). The strength of each signal was normalized to the corresponding β-actin stain signal. Data are expressed as a ratio between the protein and the corresponding β-actin signal density.

### Example 6

### Myocardial Adenosine Levels

Adenosine was analyzed by high performance liquid chromatography (HPLC) according to the procedure of Wojcik and Neff {Wojcik, 1982 #325}. Myocardial samples were homogenized with approximately 10 volumes of 0.25 M ZnSO₄. The protein concentration was determined by Lowry assay. Ten volumes of 0.25 M Ba(OH)₂ was added and the samples were centrifuged at 30,000×g for 10 minutes. The supernatants were then transferred to a conical tube and 5 μl of chloroacetaldehyde added to 20 μl of supernatant. The tubes were capped, mixed and submerged in a boiling water bath for 10 min. The samples were analyzed by HPLC using a Waters C18 reversed phase 150 mm×4.6 mm column. The mobile phase was a 50 mM acetate buffer (pH 4.5) and 6.5% aqueous acetonitrile (volume/volume) with 2 mM sodium octyl sulfonate dissolved in it. The flow rate was 1.1 ml/min. The excitation monochromator was set to a wavelength of 270 nm and the cutoff wavelength of the emission filter was 389 nm.

### Example 7

### Protein Kinase A (PKA) Activity

Myocardial samples were homogenize in 1 ml cold extraction buffer [20 mM Tris-HCl (pH7.4), 0.5 mM EDTA, 0.5 mM EGTA, 10 mM β-mercaptoethanol, 1 mM phenylmethylsulfonyl fluoride, 0.1 mM dithiothreitol (DTT), 1 μg/ml leupeptin, 1 μg/ml aprotinin, 0.1% TritonX-100], centrifuged at 14,000×g for 15 min at 4° C., and the supernatants were collected. PKA activity was measured using a cAMP-dependent protein kinase assay kit (Promega, Madison, Wis., USA,) according to the manufacturer's instructions. Briefly, the kinase reaction mixture [0.5 mM ATP, 10 μCi[γ-³²P]ATP(3,000 Ci/mmol), 0.025 mM cAMP, PKA Biotinylated Peptide Substrate, 5×PKA Assay Buffer] was prepared. The samples were diluted 2- to 16-fold in 0.1 mg/ml BSA. Then, 25 μl kinase reaction mixture was supplemented with 5 μl diluted enzyme sample (5.0 μg protein/μl) and incubated for 5 min at 30° C. Then, 10 μl samples were transferred onto a pre-numbered SAM Biotin Capture membrane Square. The membrane was washed 4 times with 2M NaCl, 4 times with 0.75% H₃PO₄ and once with deionized water. The amount of radioactivity trapped on the P81 phosphocellulose paper was quantified using a liquid scintillation counter.

### Example 8

### Statistical Analyses

Data are expressed as mean±SEM. Comparisons among the groups were performed by one-way ANOVA with Sidak correction for multiple comparisons (SPSS ver. 14.0). The differences in heart rate (HR) and mean blood pressure (MBP) were compared using two way repeated measures ANOVA with Holm-Sidak multiple comparison procedures. Values of P<0.05 were considered statistically significant.

### Example 9

### Infarct Size

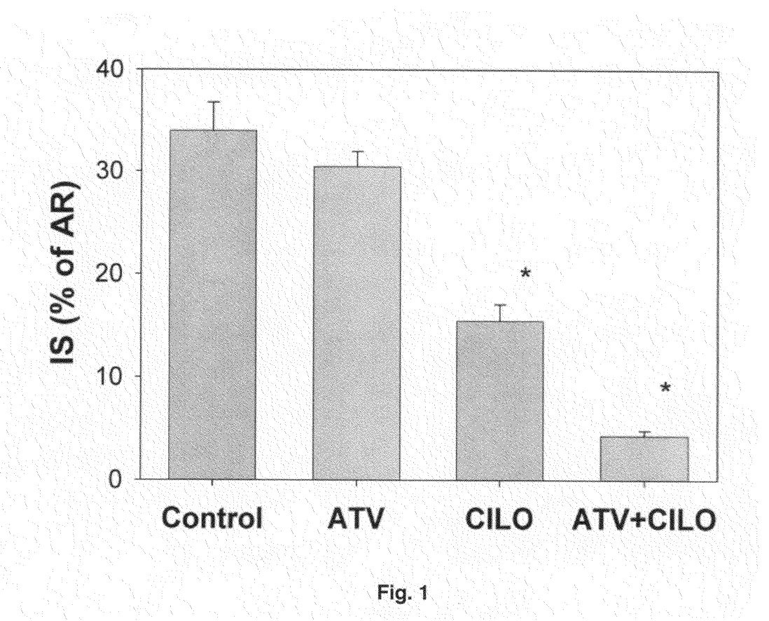

A total of 32 rats were included in the infarct size protocol (4 in each group). None of the were excluded. Body weight, left ventricular weight and the size of the AR were comparable among groups (Table 1). atorvastatin alone did not affect infarct size. cilostazol alone caused a significant reduction in infarct size (Table 1, FIG. 1). infarct size in the atorvastatin+cilostazol group was significantly smaller than in all other three groups.

TABLE 1
Protocol 1: Body weight, left ventricular (LV) weight,
area at risk (AR) and infarct size of the rats.

| | | | | atorvastatin + | |
|---|---|---|---|---|---|
| | Control | atorvastatin | Cilostazol | cilostazol | |
| | n = 8 | n = 8 | n = 8 | n = 8 | P value |
| Body | 251 ± 1 | 249 ± 1 | 248 ± 1 | 248 ± 3 | 0.389 |
| weight (g) | | | | | |
| LV (mg) | 1098 ± 34 | 1129 ± 8 | 1139 ± 3 | 1155 ± 8 | 0.205 |
| AR (mg) | 406 ± 16 | 388 ± 7 | 381 ± 12 | 379 ± 11 | 0.349 |
| infarct size | 136 ± 9 | 128 ± 8 | 56 ± 4* | 16 ± 2* | <0.001 |
| (mg) | | | | | |

*p < 0.001 versus controls.

### Example 10

### Hemodynamics

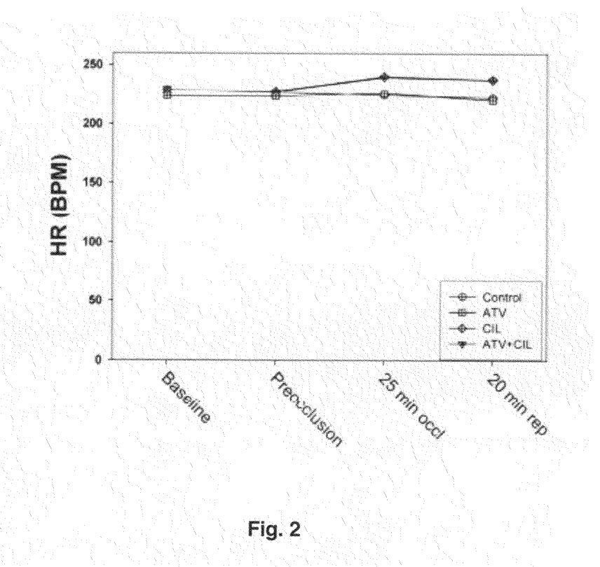

At baseline, mean heart rate was significantly slower in the atorvastatin group (223.5±1.4 bpm) than in the control (228.5±2.9 bpm; p=0.013), cilostazol (228.8±0.49 bpm; p=0.009), and atorvastatin+cilostazol group (228.5±0.5 bpm; p=0.010); however, the differences were small (FIG. 2). Just before coronary artery occlusion heart rate remained slower in the atorvastatin group (223.0±1.6 bpm) than in the cilostazol (228.0±0.4 bpm; p=0.009) and atorvastatin+cilostazol (227.5±0.4 bpm; p=0.010) group; however, it was not different from the control group (226.5±0.5 bpm; p=ns). Heart rate at 25 min of coronary artery occlusion in the control group was 224.1±0.5 bpm. Heart rate during occlusion was significantly faster in the cilostazol (241±0.8 bpm; p=0.013) and atorvastatin+cilostazol (237.8±0.3 bpm; p=0.017) groups, but not significantly different from the atorvastatin group (226.0±1.6 bpm). At 20 min of reperfusion heart rate in the control group (222.2±0.4 bpm) and atorvastatin group (219.9±1.1 bpm) were comparable. In contrast, heart rate in the cilostazol (238.0±0.7 bpm; p=0.013) and atorvastatin+cilostazol (237.8±0.3 bpm; p=0.017) were significantly faster than in the control group (FIG. 2).

Mean blood pressure (MBP) is shown in FIG. 3. Overall, there were a significant group (p=0.011), as well as time (p<0.001) effects; however, the absolute differences among groups were small. At baseline, mean blood pressure was lower in the atorvastatin group (133.9±3.4 mmHg) than in the control (139.0±0.3 mmHg; p=0.013), cilostazol (139.6±0.4 mmHg; p=0.010) and atorvastatin+cilostazol (139.9±0.9 mmHg; p=0.009) groups. Before coronary artery occlusion mean blood pressure was lower in the atorvastatin (134.0±2.0 mmHg) than in the atorvastatin+cilostazol (139.1±0.8 mmHg; p=0.009) group. During occlusion mean blood pressure was significantly lower in the cilostazol group (90.1±0.9 mmHg) than in the control group (95.4±0.9 mmHg; p=0.009) with no other significant differences among groups. There were no significant differences in mean blood pressure among groups at 20 min of reperfusion (FIG. 3).

### Example 11

### PKA Activity

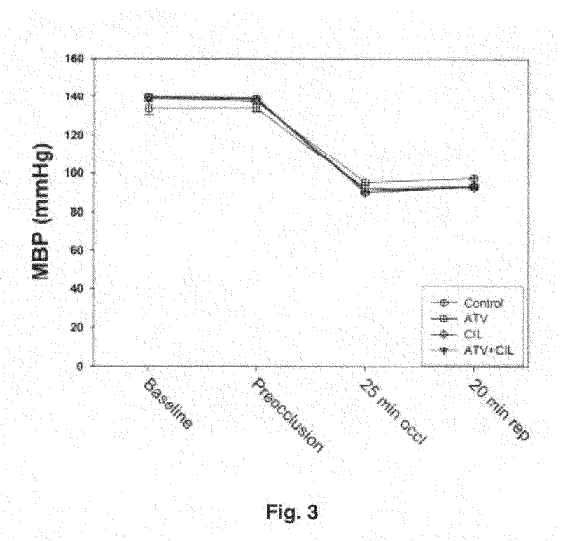

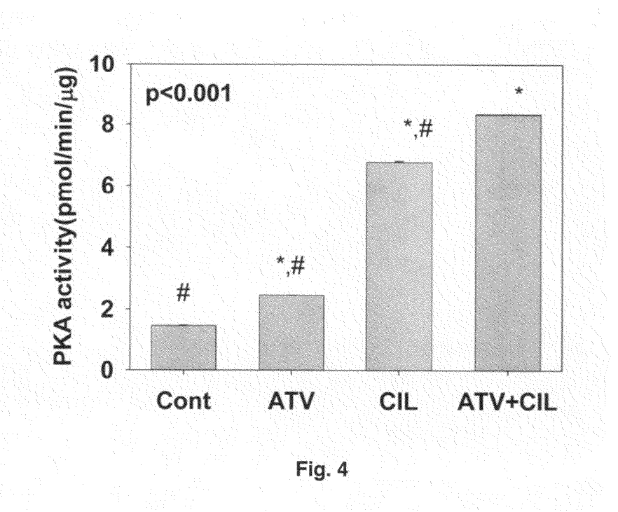

cAMP-dependent protein kinase (PKA) activity was significantly increased in the cilostazol and atorvastatin+cilostazol groups; the effect of atorvastatin alone was much smaller (FIG. 4). PKA activity levels were significantly higher in the atorvastatin+cilostazol group than in the other three groups.

### Example 12

### Myocardial Adenosine Levels

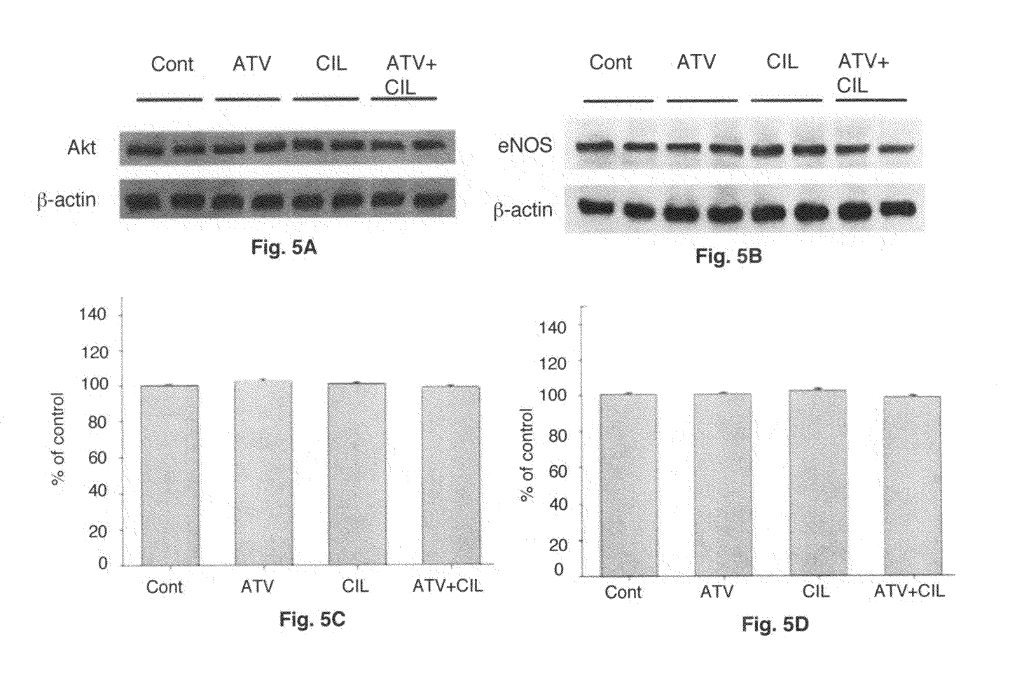

Both atorvastatin and CIL alone caused a small increase in myocardial adenosine levels. Myocardial adenosine levels in the atorvastatin+CIL combination group were significantly higher that that of the other three groups (FIG. 5).

### Example 13

### Immunoblotting

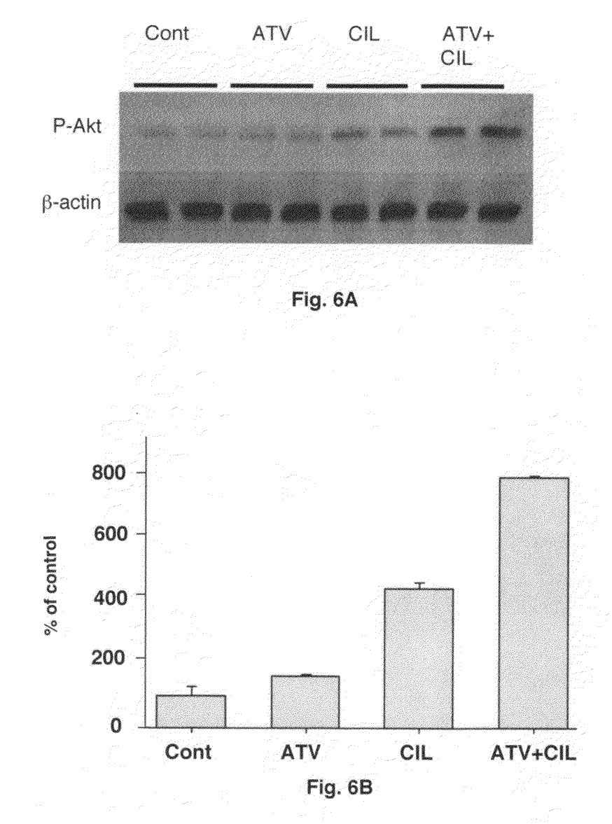

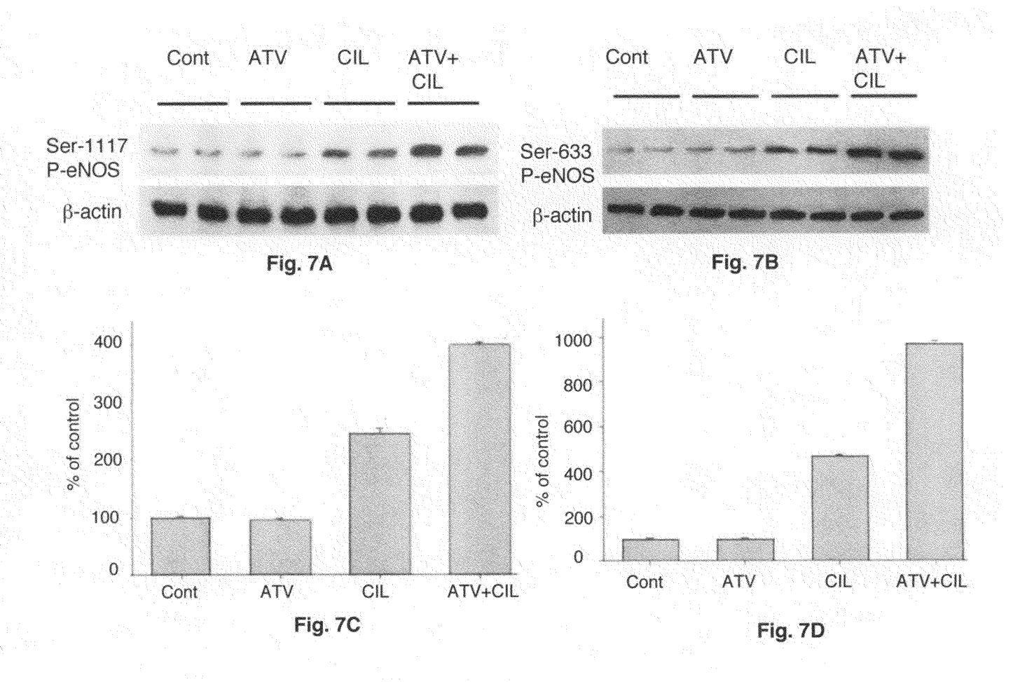

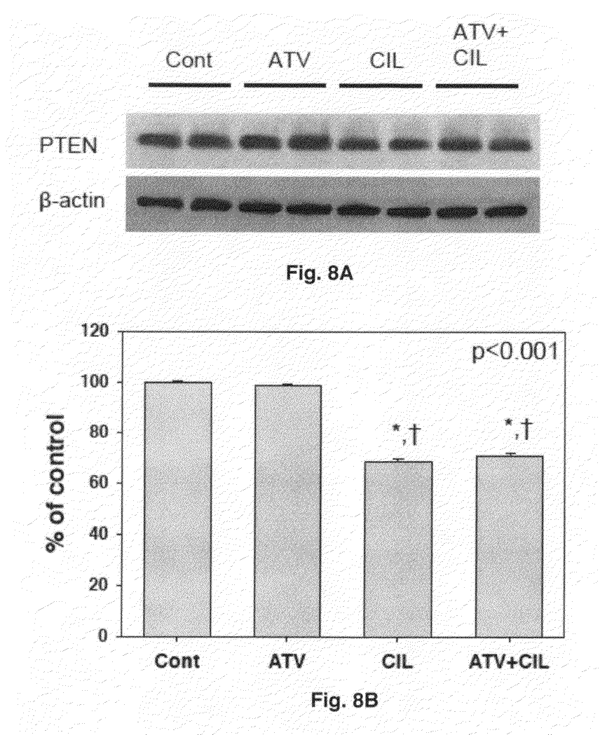

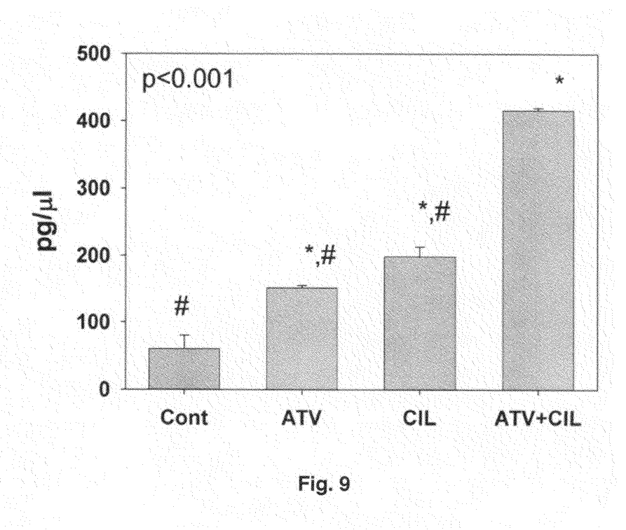

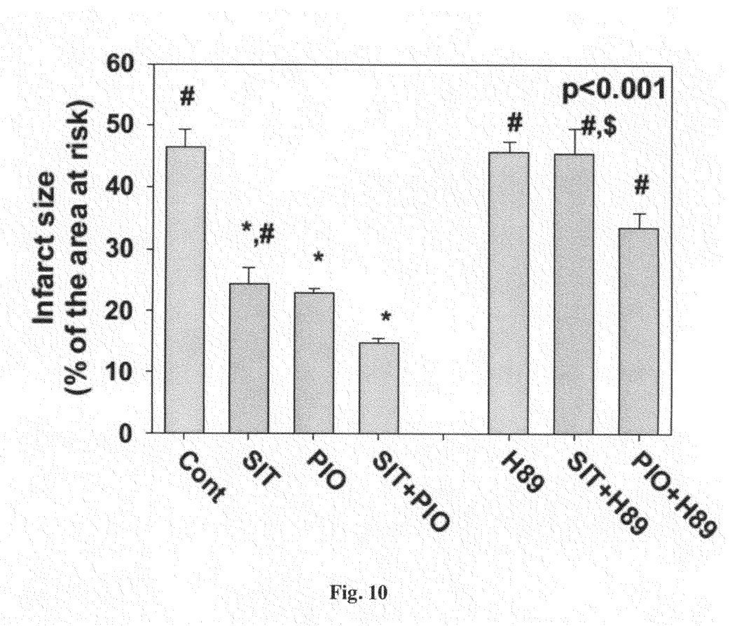

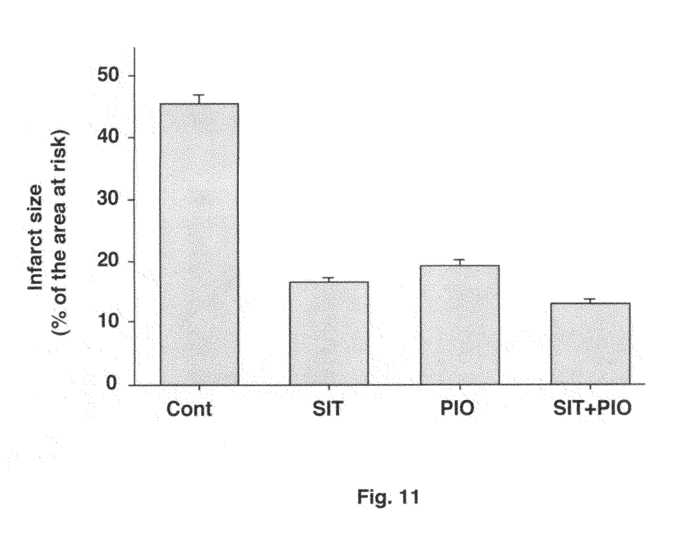

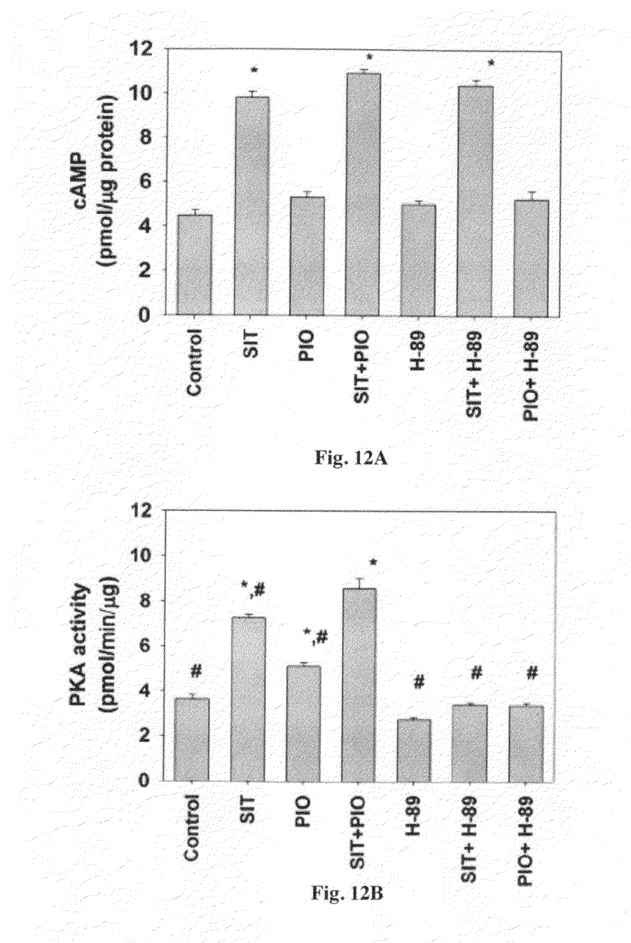

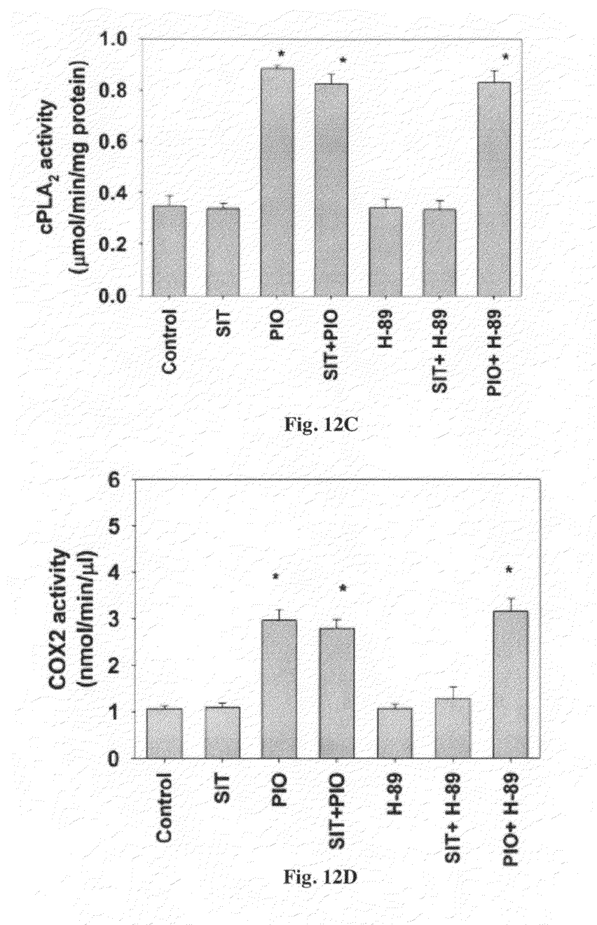

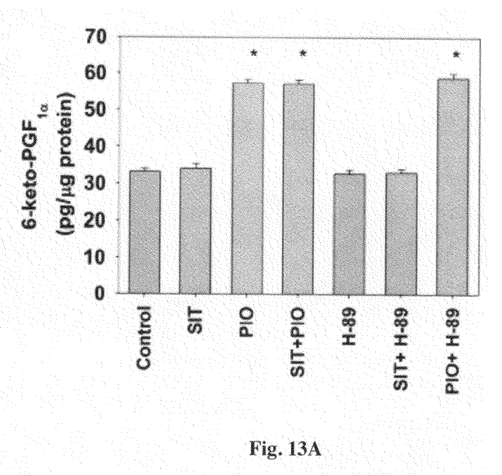

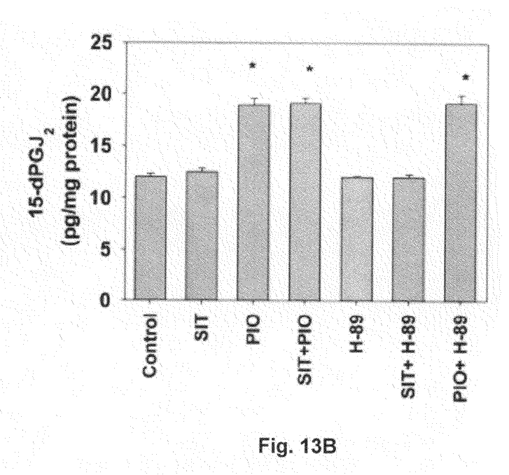

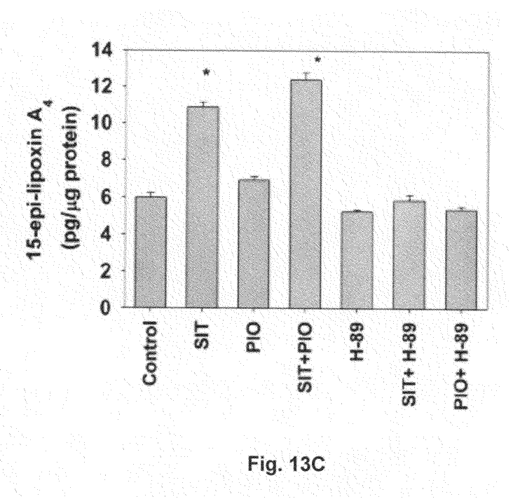

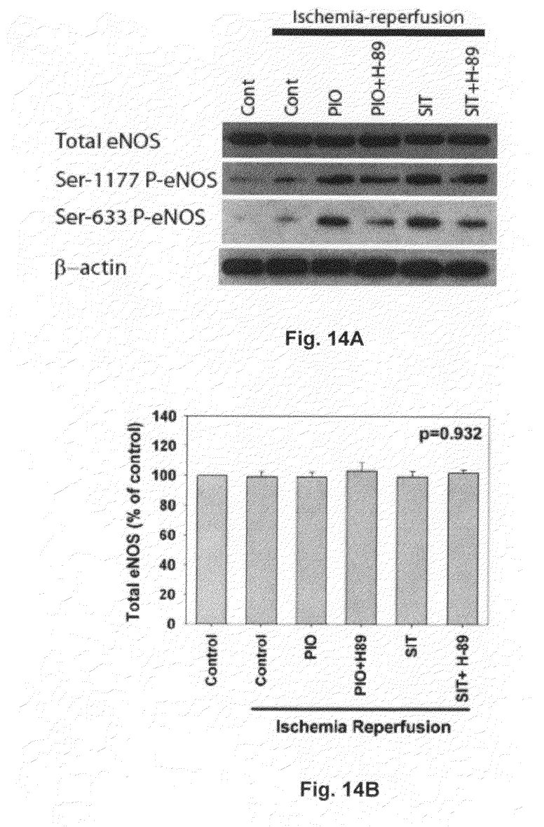

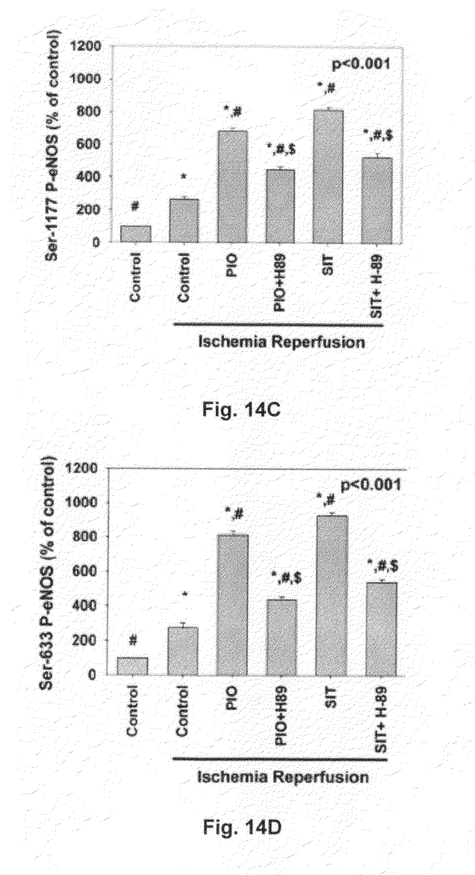

Atorvastatin and CIL, alone or in combination did not affect myocardial total Akt (FIG. 6 a and b) and total eNOS (FIG. 6 c and d) levels. Atorvastatin alone had a small, statistically significant effect on Ser-473 P-Akt levels (FIG. 7), but not on Ser-1177 P-eNOS and Ser-633 P-eNOS levels (FIG. 8). In contrast, CIL significantly increased myocardial levels of Ser-473 P-Akt (FIG. 7), Ser-1177 P-eNOS (FIG. 8 a and b) and Ser-633 P-eNOS (FIG. 8 b and c). Myocardial levels of Ser-473 P-Akt, Ser-1177 P-eNOS and Ser-633 P-eNOS were significantly higher in the ATV+CIL group than in all other three groups. Atorvastatin alone had no effect on myocardial PTEN levels (FIG. 9). CIL alone or in combination with ATV significantly decreased myocardial levels of PTEN.

### Example 14

### JANUVIA and Pioglitazone Have Synergistic Effect on Infarct Size Reduction by Activating PKA and Augmenting eNOS Activation

Wild type mice receive pretreatment with oral: 1) sitagliptin 20 mg/kg/d; 2) sitagliptin 40 mg/kg/d; 3) pioglitazone 5 mg/kg/d; 4) water alone; 5) sitagliptin 20 mg/kg/d+pioglitazone 5 mg/kg/d; 6) sitagliptin 40 mg/kg/d+pioglitazone 5 mg/kg/d. Treatment duration IS 1, 3 or 14 days. Then, mice undergo either infarct size protocol (30 min coronary artery ligation followed by 4 h reperfusion, area at risk assessed by blue dye and infarct size by TTC) (n=14 per group), or hearts are explanted without being subjected to ischemia for analysis of myocardial cAMP levels, PKA activity, 6-keto-PGF_{1α} (the stable metabolite of prostacycline), 15-epi-lipoxin A4, 15-dPGJ₂, COX2 activity, COX2 expression, total Akt and phosphorylated-Akt expression, total and phosphorylated 5-lipoxygenase expression, PKA, total eNOS, Ser-1177 P-eNOS and Ser-633 P-eNOS expression (n=6 per group). The magnitude of protection and the expression and activity of the various enzymes after 1, 3 and 14 days of pretreatment is compared to assess whether the effect decay over time or different mechanisms are activated over time, as has been suggested for ischemic preconditioning and the effect of statins.

### Methods and Materials:

Male CD-1 mice were purchased from Charles River Laboratories (Wilmington, Mass.) and received humane care. cAMP-dependent protein kinase A (PKA) assay kit was purchased from Promega (Madison, Wis.). ELISA kits for 6-keto-PGF_{1α}, and cPLA₂ and COX activity were purchased from Cayman Chemicals (Ann Arbor, Mich.); ELISA kit for 15-epi-LXA₄ from Oxford Biomedical Research (Oxford, Mich.); ELISA kit for 15DPGJ₂ and EIA for cyclic AMP levels from Assay Designs (Ann Arbor, Mich.). PIO was provided by Takeda Pharmaceuticals North America, Inc. (Lincolnshire, Ill.) and SIT by Merck. H-89, monoclonal anti-β Actin antibodies and monoclonal anti-myosin antibodies were purchased from Sigma (St. Louis, Mo.). Anti-eNOS, Ser-633 P:-eNOS, Ser-1177 PeNOS, CREB, and Ser-133 P-CREB antibodies from Cell Signaling (Beverly, Mass.).

### Treatment

Protocol 1: Mice received 3-day pretreatment with: 1) SIT (300 mg/kg per day); 2) PIO (5 mg/kg per day); 3) SIT+PIO; or water alone (control). Drugs were suspended in water and administered by oral gavage once daily. On the 4th day, mice received intravenous H-89 (20 mg/kg) or vehicle (5% DMSO) (40). One hour after injection, mice underwent coronary artery ligation for 30 minutes followed by 4-hour reperfusion, or mice were euthanized under anesthesia and hearts were explanted without being subjected to ischemia. For immunoblotting and enzyme activity, hearts were rinsed in cold PBS (pH 7.4), containing 0.16 mg/ml heparin to remove red blood cells and clots, frozen in liquid nitrogen and stored at −80° C. for further analyses.

Protocol 2: Mice received 14-day pretreatment with: 1) SIT (300 mg/kg per day); 2) PIO (5 mg/kg per day); 3) SIT+PIO; or water alone (control), as above. Mice underwent coronary artery ligation for 30 minutes followed by 4-hour reperfusion. On the fourth (protocol 1) or 15ₜₕ 142 (protocol 2) day, mice were anesthetized with intraperitoneal injection of ketamine (60 mg/kg) and xylazine (6 mg/kg), intubated and ventilated (FIO₂=30%). The rectal temperature was monitored and body temperature was maintained between 36.7 and 37.3₀ 145 C throughout the experiment. The chest was opened and the left coronary artery was encircled with a suture and ligated for 30 minutes. Ischemia was verified by regional dysfunction and discoloration of the ischemic zone. Isofluorane (1-2.0% titrated to effect) was added after the beginning of ischemia to maintain anesthesia. At 30 minutes of ischemia, the snare was released and myocardial reperfusion was verified by change in the color of the myocardium. Subcutaneous 0.1 mg/kg buprenorphine was administered, the chest was closed and the mice recovered from anesthesia. Four hours after reperfusion the mice were re-anesthetized, the coronary artery was reoccluded, Evan's blue dye 3% was injected into the right ventricle and the mice were euthanized under deep anesthesia (38, 39). The pre-specified exclusion criteria were lack of signs of ischemia during coronary artery ligation, lack of signs of reperfusion after release of the snare, prolonged ventricular arrhythmia with hypotension, and area at risk ≦510% of the left ventricular weight.

### Determination of Area at Risk (AR) and Infarct Size (IS)

Hearts were excised and the left ventricle was sliced transversely into 6 sections. Slices were incubated for 10 minutes at 37 C in 1% buffered (pH=7.4) 2,3,5-triphenyl tetrazolium-chloride (TTC), fixed in a 10% formaldehyde and photographed in order to identify the AR (uncolored by the blue dye), the IS (unstained by TTC), and the non ischemic zones (colored by blue dye). The area of AR and IS in each slice were determined by planimetry, converted into percentages of the whole for each slice, and multiplied by the weight of the slice. The results were summed to obtain the weight of the myocardial AR and IS (38, 39).

cAMP Levels and Protein Kinase A (PKA) Activity

Myocardial samples from the anterior wall of the left ventricle of hearts that were not subjected to ischemia were homogenize in 1 ml cold extraction buffer [20 mM Tris-HCl (pH7.4), 0.5 mM EDTA, 0.5 mM EGTA, 10 mM β-mercaptoethanol, 1 mM phenylmethylsulfonyl fluoride, 0.1 mM dithiothreitol (DTT), 1 μg/ml leupeptin, 1 μg/ml aprotinin, 0.1% TritonX-100], centrifuged at 14,000×g for 15 min at 4° C., and the supernatants were collected. cAMP levels and PKA activity were measured using assay kits according to the manufacturer's instructions.

6-keto-PGF_{1α}, 15dPGJ₂, 15-epi-lipoxinA₄ Levels, and Phospholipase A₂ (PLA₂) and COX2 Activity

Myocardial samples of the anterior wall of the left ventricle were homogenized in cold PBS (pH 7.4), and centrifuged. The supernatants were collected and stored on ice. Measurement of 6-Keto-PGF1α, the stable metabolite of prostacyclin, 15dPGJ₂, 15-epilipoxinA₄, and PLA₂ activity were made using immunoassay assay kits. The COX activity assay kit measures the peroxidase activity of COX, assayed colorimetrically by monitoring the appearance of oxidized N,N,N″,N″-tetramethyl-p-phenylenediamaine (TMPD) at 590 nm. Each myocardial sample was tested in triplicate (the first without an inhibitor; the second with DuP-697, a specific COX2 inhibitor; and the third with Sc560, a specific COX1 inhibitor. COX1 activity was calculated as the difference between total COX activity in the sample without an inhibitor and the sample with Sc560, and COX2 activity as the difference between total COX activity in the sample without an inhibitor and the sample with DuP-697.

### Immunoblotting

Myocardial samples from the risk zone of the anterior wall of the left ventricular wall exposed to ischemia-reperfusion (IR), or from the anterior wall of control hearts not exposed to ischemia were homogenized in lysis buffer (in mMol): 25 Tris.HCl (pH 7.4), 0.5 EDTA, 0.5 EGTA, 1 phenylmethylsulfonyl 193 fluoride, 1 dithiothreitol, 25 NaF, 1 Na₃VO₄, 1% Triton X-100, 2% SDS and 1% 194 protease inhibitor cocktail. The lysate was centrifuged at 10,000 g for 15 min at 4° C. The resulting supernatants were collected. Protein (50 μg) was fractionated by SDS-PAGE (4%-20% polyacrylamide gels) and transferred to PVDF membranes (Millipore, Bedford, Mass.). The membranes were incubated overnight at 4₀198 C with primary antibodies. Bound antibodies were detected using the chemiluminescent substrate (NEN Life Science Products, Boston, Mass.). The protein signals were quantified 200 with an image-scanning densitometer, and the strength of each 201 protein signal was normalized to the corresponding β-actin signal. Data are expressed as percent of the expression in the 202 control group.

### Statistical Analysis

Data are presented as mean±SEM. The significance level α is 0.05. Body weight, left ventricular weight, the size of the AR and IS, enzyme activity, eicosanoid levels and protein expression were compared using analysis of variance (ANOVA) with Sidak corrections for multiple comparisons.

### Results

### Infarct Size

### Protocol 1:

A total of 70 mice were included, 3 died (one in the PIO group died during ischemia and two in the SIT+H-89 died before coronary artery occlusion). Body weight, left ventricular weight and the size of the AR were comparable among groups. IS, expressed as a percent of the left ventricle or a percent of the area at risk (FIG. 1) was significantly smaller in the SIT and PIO group than in the control group. IS was the smallest in the SIT+PIO group (p<0.001 versus the control group; p=0.053 versus SIT; p=0.288 versus PIO). H-89 alone had no effect on IS; however, it completely blocked the effect of SIT whereas it only partially blocked the effect of PIO.

### Protocol 2:

A total of 32 mice were included, none excluded or died. Body weight, left ventricular weight and the size of the ischemic AR were comparable among group. IS, expressed as a percent of the left ventricle or a percent of the area at risk (FIG. 2) was significantly smaller in the SIT and PIO group than in the control group. IS was the smallest in the SIT+PIO group (p<0.001 versus the control and PIO groups; p=0.014 versus SIT group).

cAMP Levels and PKA Activity

SIT, but not PIO induced a significant increase in myocardial cAMP. H-89 alone had no effect on cAMP levels and did not block the SIT effect (FIG. 3a). Both SIT and PIO augmented PKA activity. PKA activity was significantly higher in the SIT+PIO group than in the control (p<0.001), SIT (p=0.004), and PIO (p<0.001) groups. H-89 completely blocked the SIT (p<0.001) and PIO (p<0.001) induced increase in PKA activity (FIG. 3b).

cPLA₂ Activity

PIO, but not SIT, augmented cPLA₂ activity. H-89 alone or in combination with SIT had no effect on cPLA₂ activity. H-89 did not block the effect of PIO on cPLA₂ activity (FIG. 3c).

### COX Activity

There were no significant differences among groups in COX1 activity (p=0.086). SIT had no effect on COX2 activity (FIG. 3d). In contrast, PIO significantly increased COX2 activity. H-89 alone or in combination with SIT had no effect on COX2 activity and it did not block the PIO induced increase in COX2 activity.

### Eicosanoid Levels

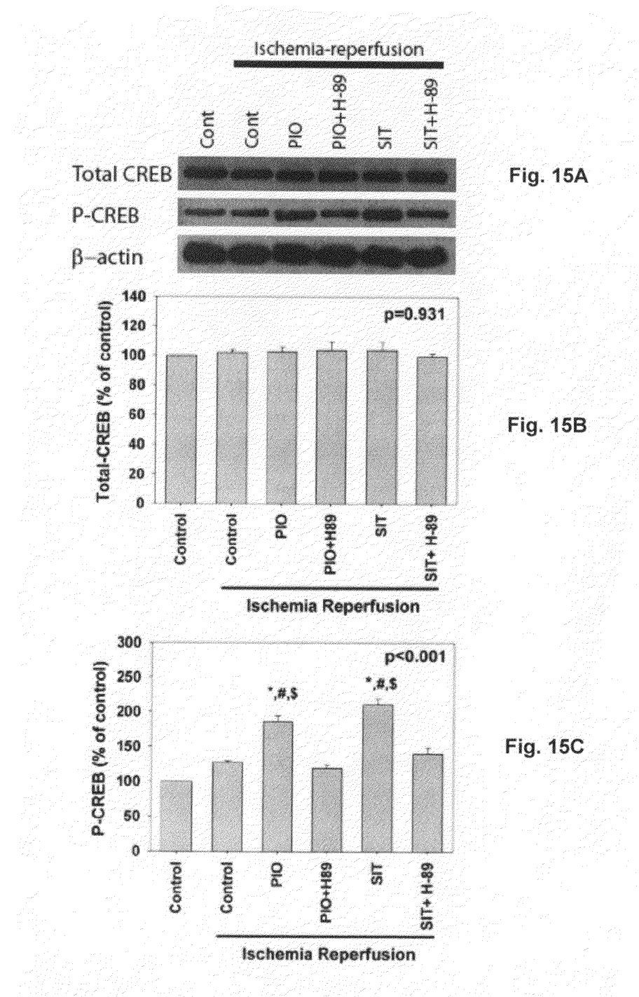

SIT had no effect on 6-keto-PGF_{1α} (FIG. 4a) or 15d PGJ₂ (FIG. 4b) levels. On the other hand, PIO increased these levels. Levels of 6-keto-PGF_{1α} and 15dPGJ₂ were comparable in the PIO alone group and the SIT+PIO group. H-89 did not block the effect of PIO. In contrast, SIT significantly increased 15-epi-lipoxinA₄ levels. PIO caused a small insignificant increase in 15-epi-lipoxinA₄ levels (FIG. 4c). 15-epi-lipoxinA₄ levels were the highest in the SIT+PIO group (p<0.001 vs. control and PIO; P=0.007 vs. SIT). H-89 completely blocked the effect of both PIO and SIT.

### Immunoblotting

For control, we used myocardial samples from mice treated with oral saline for 3 days and not exposed to ischemia-reperfusion. IR did not affect total eNOS levels. PIO and SIT had no effect on total eNOS levels (FIGS. 5a and b). IR induced an increase in Ser-1177 P-eNOS levels (FIGS. 5a and c). PIO and SIT augmented this increase, H-89 attenuated the effects of both SIT and PIO, suggesting that PKA is involved in SIT- and PIO-induced eNOS phosphorylation at Ser-1177. Similarly, IR increased myocardial levels of Ser-633 P-eNOS. Both PIO and SIT augmented this increase. H-89 attenuated this increase, suggesting that PKA is involved also in the augmented phosphorylation of eNOS at Ser-633 by both SIT and PIO.

IR without or with PIO or SIT did not affect total CREB levels (FIGS. 6a and b). IR induced a small insignificant increase in P-CREB. However, in mice pretreated with PIO or SIT, levels of P-CREB were significantly higher than in the control no IR or control IR groups (FIGS. 6a and c). H-89 blocked this effect of SIT and PIO.

The following references may have been cited herein:
1. Atar S et al. Am J Physiol Heart Circ Physiol. 2006; 290:H1960-8.
2. Birnbaum Y et al. Cardiovasc Drugs Ther. 2003; 17:25-30.
3. Birnbaum Y et al. Cardiovasc Res. 2005; 65:345-355.
4. Jones S et al. J Am Coll Cardiol. 2002; 40:1172-1178.
5. Jones S P et al. Arterioscler Thromb Vasc Biol. 2001; 21:2059-64.
6. Rosanio S et al. Cardiovasc Drugs Ther. 2006; 20:27-36.
7. Sanada S et al. Circulation. 2004; 110:2143-9.
8. Scalia R et al., Circulation. 2001; 103:2598-603.
9. Tavackoli S et al., Coron Artery Dis. 2004; 15:53-8.
10. Tiefenbacher C P et al. Am J Physiol Heart Circ Physiol. 2003; 285:H59-64.
11. Wayman N S et al. Med Sci Monit. 2003; 9:BR155-9.
12. Wolfrum S et al. J Cardiovasc Pharmacol. 2004; 44:348-55.
13. Wolfrum S et al. J Cardiovasc Pharmacol. 2003; 41:474-80.
14. Ye Y et al. Am J Physiol Heart Circ Physiol. 2006; 291:H1158-69.
15. Yamakuchi M et al. Circ Res. 2005; 96:1185-92.
16. Bell R M & Yellon D M et al. J Am Coll Cardiol. 2003; 41:508-15.
17. Efthymiou C A et al. J Cardiovasc Pharmacol. 2005; 45:247-52.
18. Hausenloy D J & Yellon D M. Cardiovasc Res. 2004; 61:448-60.
19. Kureishi Y et al. Nat Med. 2000; 6:1004-10.
20. Skaletz-Rorowski A et al. Cardiovasc Res. 2003; 57:253-64.
21. Brouet A et al. Circ Res. 2001; 89:866-73.
22. Mensah K et al. J Am Coll Cardiol. 2005; 45:1287-91.
23. Harris M B et al. Am J Physiol Heart Circ Physiol. 2004; 287:H560-6.
24. Ueda Y et al. J Am Coll Cardiol. 1999; 34:2120-5.
25. Birnbaum Y et al. Circ Res. 2006; 99:E26.
26. Gao F et al. Am J Physiol Heart Circ Physiol. 2000; 279:H329-38.
27. Smits P et al. Circulation. 1995; 92:2135-41.
28. Sobrevia L et al. J Physiol. 1997; 499 (Pt 1):135-40.
29. Lee T M et al. Am J Cardiol. 2001; 88:1108-13.
30. Ye Y et al., Am J Physiol Heart Circ Physiol. 2007 (in press).
31. Birnbaum Y, Circulation. 2006; 114:929-35.
32. Kambayashi J et al., Curr Pharm Des. 2003; 9:2289-302.
33. Hiatt W R et al. J Vasc Surg. 2002; 36:1283-91.
34. Hashimoto A et al., Atherosclerosis. 2006; 189:350-7.
35. Hong K W et al., J Pharmacol Exp Ther. 2003; 306:1182-90.
36. Kim K Y et al., J Pharmacol Exp Ther. 2004; 308:97-104.
37. Lee J H, Neurol Res. 2005; 27:483-92.
38. Lee J H et al., J Pharmacol Exp Ther. 2004; 308:896-903.
39. Liu Y et al. J Cardiovasc Pharmacol. 2000; 36:351-60.
40. Wakida K, Brain Res. 2006; 1116:187-93.
41. Birnbaum Y et al., Am J Physiol Heart Circ Physiol. 2007:In press.
42. Wojcik W J et al. J Neurochem. 1982; 39:280-2.
43. Amin-Hanjani S et al. Stroke. 2001; 32:980-6.
44. Endres M et al. Proc Natl Acad Sci U S A. 1998; 95:8880-5.
45. Boo Y C et al. J Biol Chem. 2002; 277:3388-96.
46. Dixit M et al. Circ Res. 2005; 97:1236-44.
47. Fulton D et al. J Pharmacol Exp Ther. 2001; 299:818-24.
48. Yayama K et al. Hypertension. 2006 48:958-64.
49. Krieg T et al. Am J Physiol Heart Circ Physiol. 2002; 283:H2322-30.
50. Li J et al. J Surg Res. 1998; 80:357-64.
51. Vials A & Burnstock G. Br J Pharmacol. 1993; 109:424-9.
52. Ye Y et al. J Am Col Cardiol. 2007; 49(Suppl. A):184A.
53. Birnbaum Y et al. Prostaglandins Other Lipid Mediat. 2007; 83:89-98.
54. Tsuchikane E et al. Am Heart J. 1998; 135:495-502.
55. Kunishima T et al. Clin Ther. 1997; 19:1058-66.
56. Sekiya M et al. Am J Cardiol. 1998; 82:144-7.
57. Ochiai M et al. Am J Cardiol. 1999; 84:1074-6, A6, A9.
58. Park S W et al. Am J Cardiol. 1999; 84:511-4.
59. Ge J et al. J Cardiovasc Pharmacol. 2005; 46:162-6.
60. Douglas J S Jr et al. Circulation. 2005; 112:2826-32.
61. Inoue T et al. J Am Coll Cardiol. 2004; 44:1408-14.
62. Lee J H et al. J Pharmacol Exp Ther. 2005; 313:502-9.
63. Saitoh S et al. Arterioscler Thromb. 1993; 13:563-70.
64. Dormandy J A et al. Secondary prevention of macrovascular events in patients with type 2 diabetes in the PROactive Study (PROspective pioglitAzone Clinical Trial In macroVascular Events): a randomised controlled trial. Lancet. 2005; 366:1279-89.
65. Mazzone T et al. JAMA. 2006; 296:2572-81.
66. Ye Y et al. Am J Physiol Heart Circ Physiol. 2006; 291:H1158-69.
67. Calnek D S et al. Arterioscler Thromb Vasc Biol. 2003; 23:52-7.
68. Hwang J et al. Am J Physiol Cell Physiol. 2005; 288:C899-905.
69. Chu K et al. Brain Res. 2006; 1093:208-18.
70. Gonon A T et al. Basic Res Cardiol. 2007; 102:80-9.
71. Kim K Y et al. J Biol Chem. 2006; 281:13503-12.
72. Birnbaum Y et al. Prostaglandins Other Lipid Mediat. 2007; 83:89-98.
73. Birnbaum Y et al. Circulation. 2006; 114:929-35.
74. Birnbaum et al. Jour of the Amer College of Cardiology 2007; 49 (Suppl A):355A.
75. Zhao Y et al., Faseb J. 2006; 20:1162-75.
76. Brunmair B et al. Diabetes. 2001; 50:2309-15.
77. Turturro F et al. Am J Physiol Cell Physiol. 2007; 292:C1137-46.
78. Polikandriotis J A et al., Arterioscler Thromb Vasc Biol. 2005; 25:1810-6.
79. Pereira M P et al., J Cereb Blood Flow Metab. 2006; 26:218-29.
80. Wynne A M et al., J Cardiovasc Pharmacol. 2005; 46:817-22.
81. Lee T M et al., Am J Physiol Heart Circ Physiol. 2003; 285:H1650-9.
82. Shimazu T et al., Stroke. 2005; 36:353-9.
83. Ito H et al., Lab Invest. 2003; 83:1715-21.
84. Xu Y, Am J Physiol Heart Circ Physiol. 2005; 288:H1314-23.
85. Mensah K et al., J Am Coll Cardiol. 2005; 45:1287-91.
86. Molavi B et al., Am J Physiol Heart Circ Physiol. 2006; 291:H687-93.
87. Hausenloy D J et al., Cardiovasc Res. 2006; 70:240-53.
88. Birnbaum Y et al., Journal of the American College of Cardiology 2007; 49 (Suppl A):316A.
89. Harris M B et al. Am J Physiol Heart Circ Physiol. 2004; 287:H560-6.
90. Doyle M E et al., Pharmacol Ther. 2007; 113:546-93.
91. Yusta B et al., Cell Metab. 2006; 4:391-406.
92. Bose A K et al., Diabetes. 2005; 54:146-51.
93. Thornberry N A et al. Curr Top Med Chem. 2007; 7:557-68.
   Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of reducing ischemia-reperfusion injury in an individual in need of such treatment comprising:
administering a compound or a combination of compounds, in an amount effective in increasing intracellular levels of cyclic adenosine monoposphate, thereby reducing ischemia-reperfusion injury in said individual.

2. The method of claim 1, wherein said administration activates protein kinase A.

3. The method of claim 2, wherein said activation of protein kinase A induces activation of endothelial nitric oxide synthase via phosphorylation of Ser-633 and Ser-1177 residues of the endothelial nitric oxide synthase polypeptide.

4. The method of claim 1, wherein said individual at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy.

5. The method of claim 4, wherein the treatment results in reduction of infarct size in said individual.

6. The method of claim 1, wherein said compound is a dipeptidyl peptidase-4 inhibitor.

7. The method of claim 6, wherein said dipeptidyl peptidase-4 inhibitor is vildagliptin, sitagliptin or saxagliptin.

8. The method of claim 1, wherein said compound is a ligand for the peroxisome proliferator-activated receptor gamma.

9. The method of claim 8, wherein said peroxisome proliferator-activated receptor gamma is a thiazolidinediones.

10. The method of claim 9, wherein said thiazolidinediones is pioglitazone or rosiglitazone.

11. The method of claim 1, wherein said combination of compounds administered is a combination of a dipeptidyl peptidase-4 inhibitor and a thiazolidinedione.

12. The method of claim 1, wherein said compound is a phosphodiesterase type three inhibitor.

13. The method of claim 12, wherein said phosphodiesterase type 3 inhibitor activates endothelial nitric oxide synthase activation via phosphorylation of Ser-633 and Ser-1177 residues of the endothelial nitric oxide synthase polypeptide.

14. The method of claim 13, wherein said phosphorylation of Ser-633 and Ser-1177 residues of endothelial nitric oxide synthase involves activation of AKT and protein kinase A and suppression of PTEN activity.

15. The method of claim 13, wherein said endothelial nitric oxide synthase activation is due to suppression of adenosine reuptake into the cell.

16. The method of claim 12, wherein the phosphodiesterase 3 inhibitor is cilostazol, amrinone, bucladesine, enoximone or milrinone.

17. A method of augmenting the cardioprotective effects of a HMG-CoA reductase inhibitor in an individual comprising:
co-administering pharmacologically effective amounts of a HMG CoA reductase inhibitor with a phosphodiesterase 3 inhibitor, wherein said co-administration augments the cardioprotection in the individual.

18. The method of claim 17, wherein said co-administration of pharmacologically effective amounts of HMG CoA reductase inhibitor and the phosphodiesterase 3 inhibitor lead to a synergistic activation of endothelial nitric oxide synthase.

19. The method of claim 17, wherein the treatment results in reduction of infarct size in said individual.

20. The method of claim 17, wherein said individual at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy.

21. The method of claim 17, wherein said HMG-CoA reductase inhibitors are selected from the group consisting of atorvastatin, cerivastatin, fluvastatin, lovastatin, mevastatin and pitastatin.

22. The method of claim 17, wherein said phosphodiesterase 3 inhibitor is cilostazol, amrinone, bucladesine, enoximone or milrinone.

23. A method of augmenting the cardioprotective effects of a HMG-CoA reductase inhibitor in an individual comprising:
co-administering pharmacologically effective amounts of a HMG CoA reductase inhibitor with a compound effective in increasing intracellular levels of cyclic adenosine monoposphate, wherein said co-administration augments the cardioprotection in the individual.

24. The method of claim 23, wherein said individual at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, or myocardial hypertrophy.

26. The method of claim 23, wherein the treatment results in reduction of infarct size in said individual.

27. The method of claim 23, wherein said compound is a dipeptidyl peptidase-4 inhibitor.

28. The method of claim 27, wherein said dipeptidyl peptidase-4 inhibitor is vildagliptin, sitagliptin or saxagliptin.

29. The method of claim 23, wherein said compound is a ligand for the peroxisome proliferator-activated receptor gamma.

30. The method of claim 29, wherein said peroxisome proliferator-activated receptor gamma is a thiazolidinediones.

31. The method of claim 30, wherein said thiazolidinedione is pioglitazone or rosiglitazone.

32. The method of claim 23, wherein said HMG-CoA reductase inhibitors are selected from the group consisting of atorvastatin, cerivastatin, fluvastatin, lovastatin, mevastatin and pitastatin.
